(19) United States
(12) Patent Application Publication
(10) Pub. No.: US 2019/0090731 A1
(43) Pub. Date: Mar. 28, 2019

(54) CLEANING DENTAL MIRRORS

(71) Applicant: NOVA SOUTHEASTERN UNIVERSITY, Fort Lauderdale, FL (US)

(72) Inventor: Daniel J. Young, Fort Lauderdale, FL (US)

(73) Assignee: NOVA SOUTHEASTERN UNIVERSITY, Fort Lauderdale, FL (US)

(21) Appl. No.: 16/087,194

(22) PCT Filed: Mar. 23, 2017
(86) PCT No.: PCT/US17/23759

§ 371 Date: Sep. 21, 2018

Related U.S. Application Data
(60) Provisional application No. 62/311,953, filed on Mar. 23, 2016.

(51) Int. Cl. A61B 1/253; A61B 1/00; A61B 1/015

(52) U.S. Cl. CPC A61B 1/253; A61B 1/00091; A61B 1/015; A61B 1/00101; A61B 1/00094

(57) ABSTRACT

A cleaning tool is attachable to a dental air-water syringe tip. The tool has a frame which supports a mirrored surface. tool grip is attached to the frame, and includes a resilient body and a channel extending through the body. The channel is sized smaller than a diameter of the syringe tip. A support arm connects the frame and the tool grip, and the frame and the tool grip are each connected to the support arm at a predetermined angle which causes there to be a predetermined angle between a longitudinal axis of the channel, and thus the output of the tool tip, and the mirror surface.

### FIELD OF THE DISCLOSURE

The disclosure relates to a system and method for cleaning dental mirrors, and in particular, to using an automated air-water syringe to clean a dental mirror.

### BACKGROUND OF THE DISCLOSURE

A mouth mirror or dentist's mirror is an instrument used in dentistry, having a round head, used for indirect viewing of locations within the mouth. The dentist's mirror enables the dental practitioner to maintain good posture while viewing various areas. The mirrors commonly have sizes of 1-Ø 16 mm, 2-Ø 18 mm, 3-Ø 20 mm, 4-Ø 22 mm, 5-Ø 24 mm. The mirror surface can be flat, concave, or double sided. Numbers 4 and 5 are used most commonly, with number 2 used in smaller areas.

An air-water syringe is a tool which supplies a stream of air and/or water through a nozzle that may be inserted into the mouth during a dental procedure, typically to clean teeth surfaces. They can include quick disconnect nozzle tips, which can be autoclavable.

### SUMMARY OF THE DISCLOSURE

In an embodiment of the disclosure, a device for attachment to an air-water syringe tip comprises a frame; a mirror surface disposed upon the frame; a tool grip including a resilient body and a channel extending through the body sized smaller than a diameter of a syringe tip, the channel defining a longitudinal axis; and a support arm connecting the frame and the tool grip, the frame and the tool grip each connected to the support arm at a predetermined angle to thereby form a predetermined angle between the longitudinal axis of the channel and the mirror surface.

In variations thereof, the tool grip is fabricated from a polymeric material; the tool grip further includes a gap extending along the longitudinal axis from the channel to an exterior of the tool grip; the predetermined angle between the longitudinal axis of the channel and the mirror surface is between 25 and 70 degrees; the predetermined angle between the longitudinal axis of the channel and the mirror surface is about 45 degrees; the tool grip is rotatable 360 degrees about the tool tip when the tool tip is inserted into the channel; and/or the frame is produced by 3D printing.

In another embodiment of the disclosure, a system comprises an air-water dental tool tip for replaceable attachment to an air-water dental syringe tool; a mountable mirror connectable to the air-water dental tool tip, including: a frame; a mirror surface disposed upon the frame; a tool grip including a resilient body and a channel extending through the body sized smaller than a diameter of a syringe tip, the channel defining a longitudinal axis;

and a support arm connecting the frame and the tool grip, the frame and the tool grip each connected to the support arm at a predetermined angle to thereby form a predetermined angle between the longitudinal axis of the channel and the mirror surface.

In variations thereof, the system includes a plurality of air-water dental tool tips of differing tool tip diameters, and a plurality of mountable mirrors, at least one mountable sized for mounting to each of the plurality of tool tips by having a channel sized smaller than the diameter of the tool tip.

In other variations thereof, the tool grip is fabricated from a polymeric material; the tool grip further includes a gap extending along the longitudinal axis from the channel to an exterior of the tool grip; the predetermined angle between the longitudinal axis of the channel and the mirror surface is between 25 and 70 degrees; the the predetermined angle between the longitudinal axis of the channel and the mirror surface is about 45 degrees; the tool grip is rotatable 360 degrees about the tool tip when the tool tip is inserted into the channel; the frame is produced by 3D printing; and/or the frame is formed from a material selected from the group consisting of polylactic acid, PVC, polyethylene, PEEK, polycarbonate, Polyetherimide, Polysulfone, Polypropylene, and Polyurethane.

In another embodiment of the disclosure, a method of cleaning a dental mirror comprises providing a device attachable to an air-water syringe tip, the device including a frame; a mirror surface disposed upon the frame; a tool grip including a resilient body and a channel extending through the body sized smaller than a diameter of a syringe tip, the channel defining a longitudinal axis; a support arm connecting the frame and the tool grip, the frame and the tool grip each connected to the support arm at a predetermined angle to thereby form a predetermined angle between the longitudinal axis of the channel and the mirror surface.

In variations thereof, the tool grip is fabricated from a polymeric material; the tool grip further includes a gap extending along the longitudinal axis from the channel to an exterior of the tool grip; and/or the gap is resiliently expanded when the tool tip is inserted into the tool grip.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is a perspective view of an attachable dental mirror device of the disclosure;

FIG. 2 is a perspective view of an alternative attachable dental mirror device of the disclosure;

FIG. 3 is a perspective view of the device of FIG. 2, showing hidden lines;

FIG. 4 depicts the device of FIG. 1 mounted to an automated air-water syringe;

FIG. 5 depicts an automated air-water syringe with replaceable tool tips, and separate attachable dental mirror devices of the disclosure, each adapted to a particular tool tip of the syringe;

FIG. 6 depicts a mounting device of the disclosure for attaching a mirror head having a post or shaft to an automated air-water syringe tool tip;

FIG. 7 depicts a detailed view of the mounting device of FIG. 6;

FIG. 8 depicts a device of the disclosure in a form as just printed using a 3D printing manufacturing process;

FIG. 9 depicts the device of FIG. 8 showing hidden lines;

FIG. 10 depicts the device of FIG. 1 sized and dimensioned to securely grip a syringe tip of a low-profile pen-style air/water syringe;

FIG. 11 depicts the device of FIG. 1 mounted upon a tool tip of the pen-style air/water syringe of FIG. 10;

FIG. 12 depicts an alternative mirror frame of the disclosure;

FIG. 13 depicts a cross-section of the frame of FIG. 12;

FIG. 14 depicts a perspective hidden line view of an embodiment of the device of FIG. 6, including an inner threaded bore;

FIG. 15 depicts a cross section taken along line A-A of FIG. 16, of a dual bore tool attachment of the disclosure including an air/water injection bore and a suction bore;

FIG. 16 depicts a tool of the disclosure having the tool tip of FIG. 15;

FIG. 17 depicts the tool tip of FIGS. 16-17 inserted into a mirror frame of the disclosure;

FIG. 18 depicts an internal pressurized reservoir within a handheld tool, for release of gas or liquid from a device of the disclosure;

FIG. 19 depicts a cross section of a dual bore tool tip as described with respect to FIG. 15, and having an oval profile;

FIG. 20 depicts a mirror frame of the disclosure including peripheral suction/aspiration ports;

FIG. 21 depicts the mirror frame of FIG. 19, mounted upon the device of FIG. 16; and

FIG. 22 is a detail of a dual bore connection between the tool tip of FIG. 15 and the mirror frame of FIG. 19.

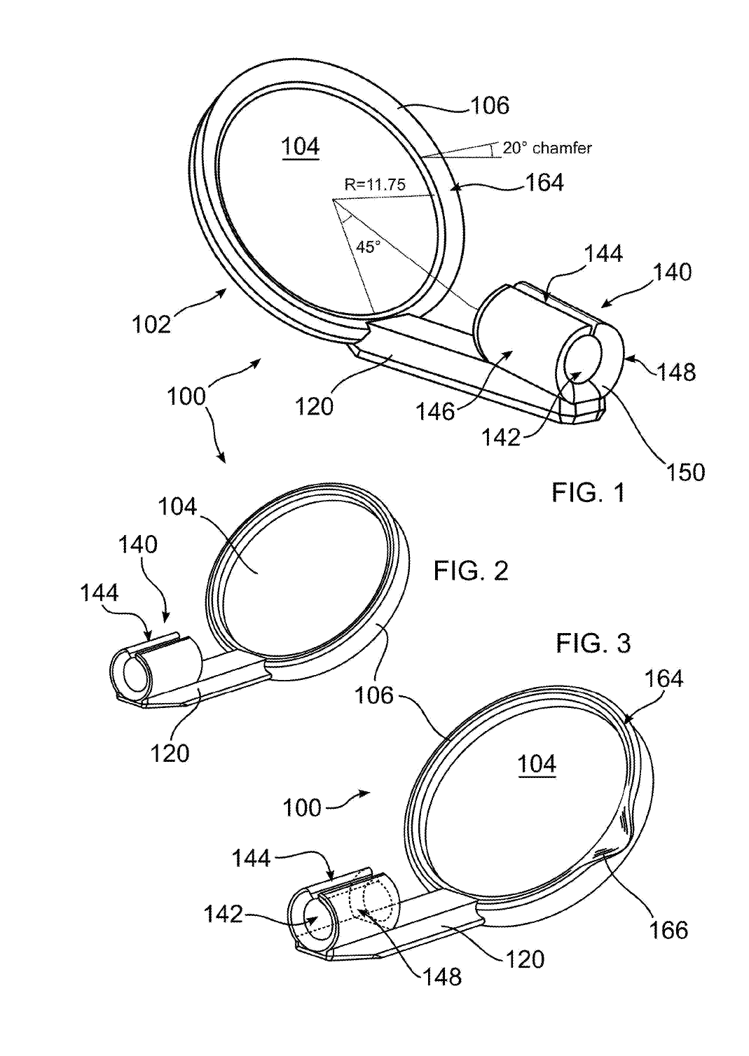

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

The disclosure provides a dental mirror that can be removably clipped or fastened to dental air and water supply equipment, or syringes, thereby adapting the mirror to be cleanable without a requirement to remove the mirror and interrupt a therapeutic dental procedure. More particularly, an attachable mirror assembly of the disclosure, or device 100, can be releasably affixed to a shaft 312 of a tool tip 310 of a prior art air or water (hereinafter ‘fluid’) spraying tool 300, whereby the jet of air or water (indicated by arrows “A”) released from shaft 310 is directed towards a mirror 102 surface 104, whereby debris is driven off surface 104 by pressure, thereby cleaning surface 104. As tool 300 is typically operated by a foot switch, a dental practitioner can case a jet of fluid to be emitted from a nozzle outlet 314 of tool tip 310 to clean surface 104, while both hands of the practitioner can continue with the therapeutic procedure. In this manner, time of the practitioner and the patient are not lost due to many repeated instances of interruption for cleaning the mirror.

Device 100 includes a mirror 102 having a reflecting mirror surface 104, a mirror supporting frame 106, a support arm 120, and a tool grip 140. A chamfer 164 can be formed in frame 106, to reduce visual obstruction to mirror surface 104. For example, a chamfer of 20 degrees, although other angles can be formed as understood within the art. Frame 106 is sized to be larger than mirror surface 104 only as required to support mirror surface 104, or can be substantially larger. In an embodiment, chamfer 164 is shaped to deflect or direct fluid at a particular angle, which has impacted mirror surface 104 and is being ejected from mirror 102. In another embodiment, channel 164 concentrates or funnels fluid in a particular direction, by forming a progressively shallower angle to form a spout 166, as shown in FIG. 3.

Mirror surface 104 can be any size useful in the mouth, including any standard size, for example a diameter of about 24 mm, although substantially larger or smaller sizes can be used, for example as small as 12 mm or less, and as large as 28 mm or more, can be provided. In an embodiment, frame 106 does not have a diameter larger than mirror surface 104.

FIG. 5 illustrates that existing and hereinafter developed syringe devices 300 include replaceable tool tips 310, an example of which is shown as tool tip 310A, which has a larger diameter than tool tip 310. It may be seen that tool grip 140A can be sized larger, or have a larger channel 142, than tool grip 140, and that devices 100 can thus be provided in different sizes to accommodate the variety of tool tip 310 shafts 312 which are commonly used in the practice of dentistry and its related arts. Further, such replaceable tool tips can include correspondingly sized devices 100, 200 as kits. It may also be seen that mirror 104 size can remain constant for the various embodiments having larger or smaller tool grip 140 or channel 142. Likewise, a given tool grip 140 can have sufficient resilience in order to provide a grip strength within a predetermined acceptable range for a variety of shaft 312 sizes.

Frame 106 is connected to support arm 120 at an angle that practitioners commonly find convenient for manipulating the mirror to view the various surfaces within the mouth, for example between 20 and 70 degrees. An angular relationship between mirror surface 104 and tool tip 310 is additionally determined by an angular disposition of a connection between tool grip 140 and arm 120, as detailed below. Other angles can be formed, or a connection between frame 106 and arm 120 can be adjustable, for example by including a bendable metallic core within arm 120 or between arm 120 and frame 106, or a detent connection can be provided between frame 106 and arm 120. Arm 120 advantageously is shaped with rounded or low angle surfaces, so that it does not introduce any discomfort to the patient when in contact with body tissue.

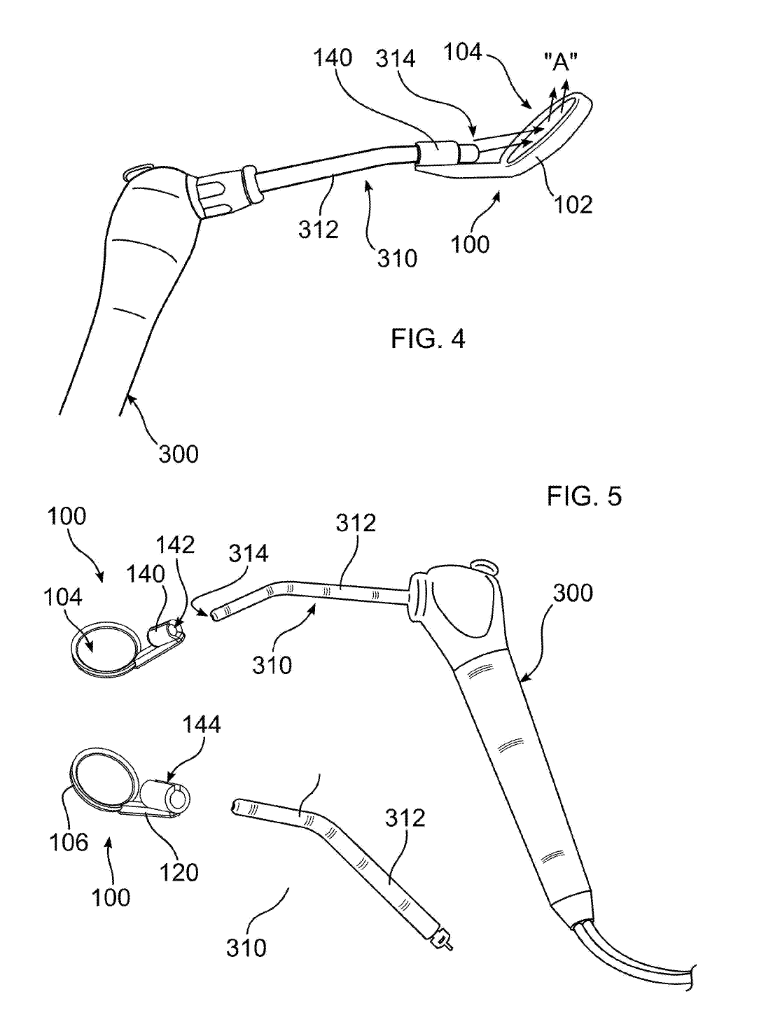

Tool grip 140 is positioned at a point along arm 120, for example at an end of arm 120. In an embodiment, tool grip 140 is positioned to direct fluid emitted from shaft 312 to mirror surface 104. In the embodiment shown, an angular disposition of tool grip 140 cooperates with an angular relationship between arm 120 and frame 106 to cause the emitted jet of fluid to strike mirror surface 104 at a low or glancing angle (arrows “A” in FIG. 4), so that the fluid and the contaminants are together displaced and ejected from mirror surface 104. In an embodiment, the angle is about 45 (+/−5) degrees, although other angles can be effective, for example an angle between 15 and 75 degrees.

To form an attachment to shaft 312 of an existing tool tip 310, tool grip is formed of a resilient material with a central channel 142 into which an end of the tool tip shaft 312 can be inserted. The central channel is sized to be smaller than diameter of shaft 312, so that a gripping or clamping force is imparted between tool grip 140 and shaft 312 as a result of the interference fit. In one embodiment, tool grip 140 forms a closed channel, whereby tool grip 140 must expand, or material of tool grip 140 must compress. In the embodiment illustrated, tool grip 140 includes a longitudinal slit or gap 144 forming an opening into channel 142, enabling sidewalls 146, 148 to resiliently separate in order to grip a range of tool tip shaft 312 sizes, and/or to facilitate insertion of shaft 312. A bevel or insertion chamfer 150 can be provided to guide shaft 312 into alignment with channel 142. Gap 144 can extend along part or all of a longitudinal axis extending through channel 142, whereby gap 144 facilitates entry of tool tip shaft 312 into channel 142, and channel 142 forms a more secure grip during a portion of tool grip 140 which does not include gap 144.

The force of grip between tool grip 140 and shaft 312 is a function of the coefficient of friction of the materials of grip 140 and shaft 312, and the clamping or interference force exerted by grip 140. In one embodiment, tool grip 140 is formed with polylactic acid, has a channel width of 2.4 mm, and sidewall 146, 148 thickness of 1.6 mm. As such, shaft 312 is gripped with sufficient force to enable mirror 102 to be used to retract soft tissue, such as the lips, gum, or tongue during a dental procedure, without device 100 moving upon shaft 312. However, the grip exerted enables the practitioner to manually remove device 100 from shaft 312, or to radially rotate device 100 upon shaft 312 to a desired orientation, without excessive difficulty.

Materials used for the various components of device 100 are selected to be biocompatible, resistant to degradation due to the substances found in the mouth and the chemicals used during dental related procedures, and are advantageously of a softness which reduces a likelihood of damage to tooth structure during use. Mirror surface can be a polished material that is biocompatible, for example a metallic surface, or metallized surface. In an embodiment, tool grip 140, frame 106, and arm 120 are molded together as a single unitary part. Alternatively, the foregoing elements can be cast, formed, 3D printed, or connected by any method, including for example adhesive or high frequency welding. In an embodiment, frame 106, arm 120, and tool grip 140 are fabricated with a polymeric substance, for example polylactic acid, PVC, polyethylene, PEEK, polycarbonate, Polyetherimide, Polysulfone, Polypropylene, and Polyurethane.

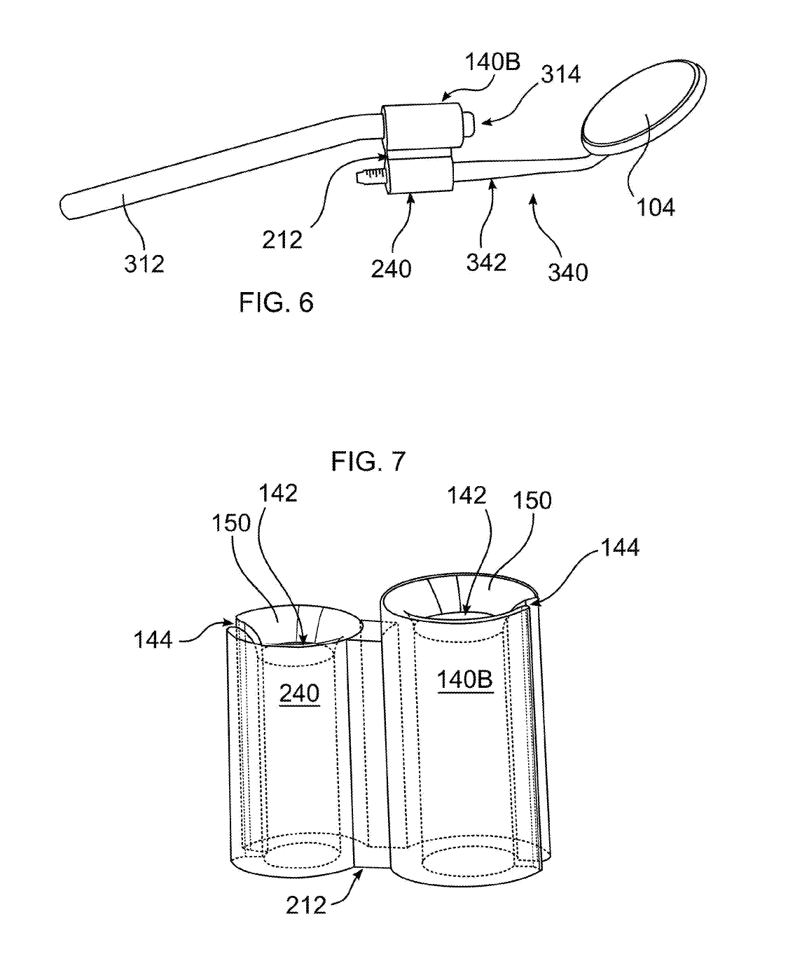

Referring now to FIGS. 6-7, connector device 200 of the disclosure includes a secondary tool grip 240, connected to a tool grip 140B which functions in a similar manner as described with respect to grip 140, for attaching to an existing tool tip shaft 312. In the embodiment of FIG. 6, a KERR SEAL-TIGHT air-water syringe tip is illustrated, removed from tool 300, although any syringe tip can be used together with device 100 or 200 of the disclosure. Tool grip 240 is sized and dimensioned to grip a preexisting dental mirror component 340, such as is shown in FIG. 6. In the example shown, an end portion of a HU-FRIEDY MH 24 mirror head has been unthreaded from an associated handle, and a mirror head shaft 342 has been inserted within tool grip 240, and is held therein in the manner as described with respect to tool grip 140.

More particularly, as shown in FIG. 7, both tool grip 240 and tool grip 140B have a longitudinal gap 144, a channel 142, sidewalls 146, 148, all of which function in the manner as described with respect to tool grip 140. Tool grips 240 and 140B are connected together along a longitudinal axis by a bridge 212, in this example forming a unitary part together with grips 240, 140B. As illustrated, bridge 212 is narrower than a diameter of either grip 240 or 140B, however it should be understood that grips 240 and 140B can be formed as separate adjacent channels within a single oval, rounded, rectangular, or polygonal block shaped profile, and that bridge 212 can therefore not appear as a discrete part.

Grips 240 and 140B form adjacent channels each holding a respective tool tip shaft 312 and mirror head shaft 342. In the example shown, grips 240 and 140B have coplanar axes, however these axes can be offset, to provide a predetermined relative angle between mirror head shaft 342 and tool tip shaft 512, with a resultant desired angular offset between mirror 104 and tool tip 310. As described with respect to grip 140, mirror head may be radially rotated within grip 240 to position mirror 104 at a desired radial angle with respect to a jet of fluid emerging from an outlet 314 of shaft 512.

Device 200 enables any mirror or other tool having a shaft connector to be attached to a tool tip shaft 512. A mirror head device, similar to that shown in FIG. 6, can be provided with device 200, and can be particularly adapted to snap fit into tool grip 240. Tool grip 240 can be provided with an insertion chamfer to align the mirror device shaft 342 into channel 142 of tool grip 240. As illustrated in FIG. 7, longitudinal gap 144 of any of tool grip 240, 140B, or 140 can be angularly formed along a longitudinal length of channel 142, to provide a more attractive, unbroken, appearance, and to provide greater initial resiliency when inserting shaft 512 or 342. To facilitate quick visual identification as to which of grip 240 or 140B is to be used to connect to either mirror head shaft 342 or tool tip shaft 312, grips 240 and 140B can be fabricated with unequal lengths. For example, grip 240 can be made shorter, to suggest a smaller channel 142 diameter for fitment to a relatively smaller diameter mirror head shaft 342. Device 200 can be fabricated in the manner, and with the materials described with respect to device 100.

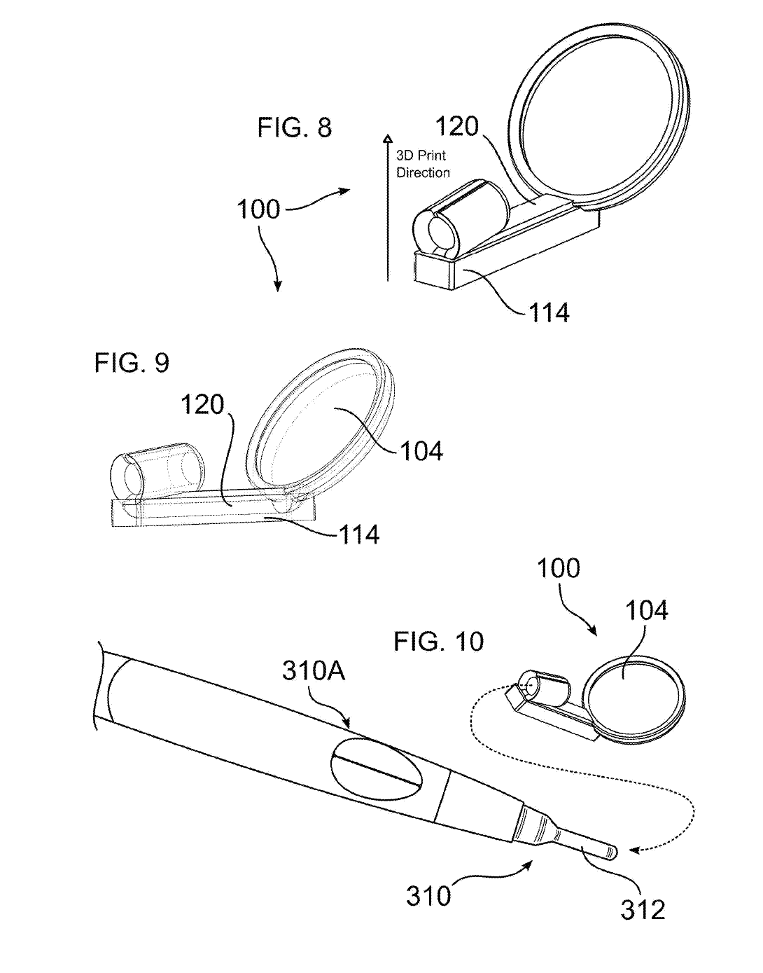

FIGS. 8 and 9 illustrate an example device 100 as completed after 3D print manufacturing. Note that there a dissolvable base mold provides print support to create round bottom edges for arm 120 during the printing process. The lower portion of arm 120 is embedded within the dissolvable support 114, as can be seen in FIG. 9. Due to the design as shown, it is possible to provide device 100 as a ready-to-print stl model, reducing distribution costs and enabling more dental practitioners to realize the benefits of the disclosure.

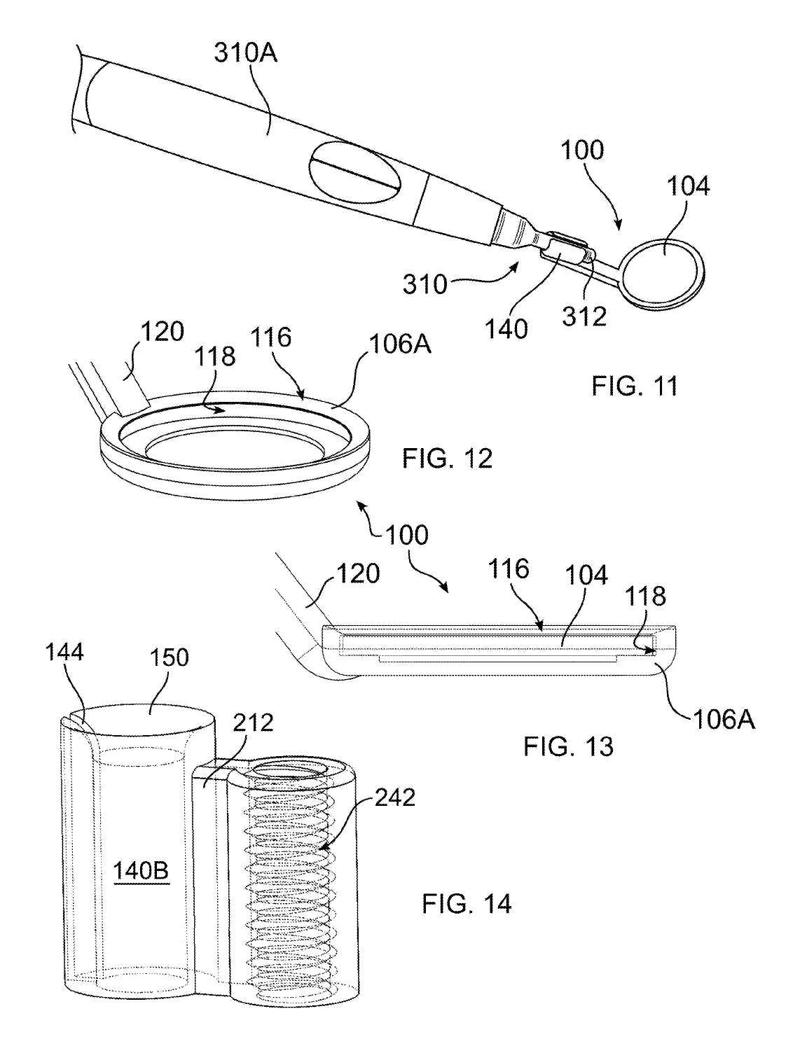

FIG. 10-11 illustrate device 100 configured for attachment to a low-profile pen-like air/water syringe, in this example a FARO SYR dental syringe 310A. The pen style grip enables control of air/water while maintaining an optimum and ergonomic grip upon the syringe tool and attached device 100. This enables maintenance of a proper viewing angle without substantial movement of the hand while activating a fluid spray onto mirror surface 104.

FIG. 12 depicts an alternative frame 106A of the disclosure, which includes a mechanical locking engagement 116 including a deformable channel 118, which can be stretched to allow insertion of a separate mirror (not shown). In this manner, device 100 can be 3D printed, for example, and a mirror can be added later without the use of adhesives. This can further be understood in view of the cross-section of FIG. 13.

FIG. 14 illustrates a threaded inner bore 242 within tool grip 240A of device 200A, so that a dental mirror component 340, for example as shown in FIG. 6, or other dental tool to be sprayed or washed with fluid can be threadably attached to device 200A.

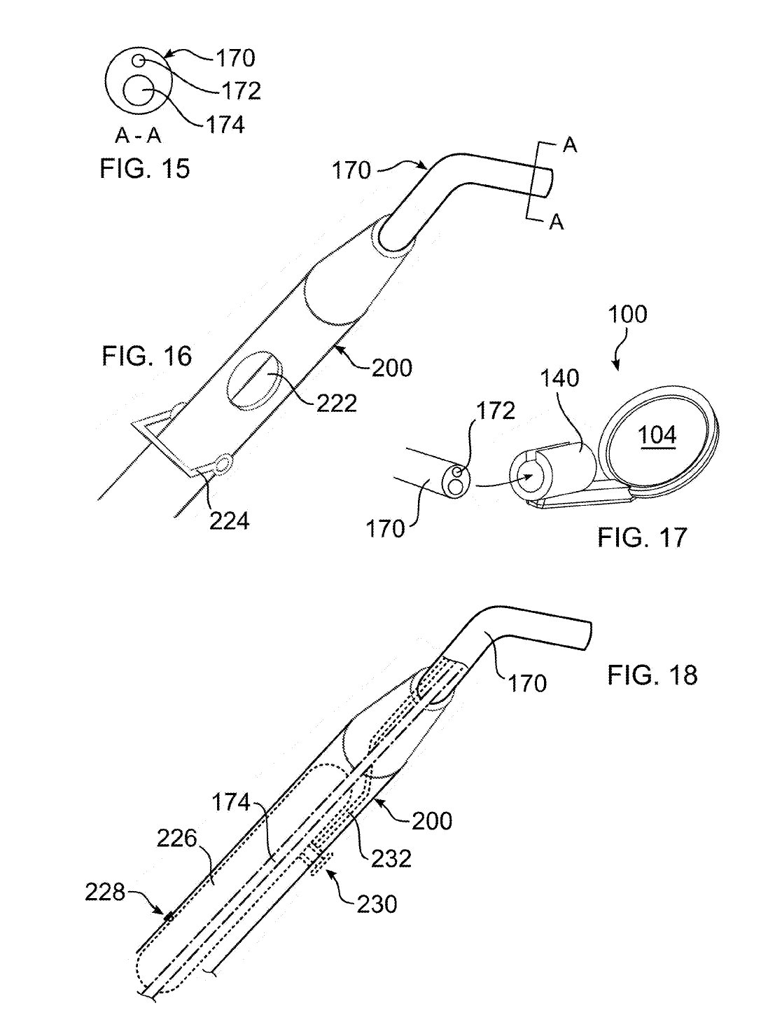

Referring now to FIGS. 15 and 16, a tool tip 170 includes a bore 172 for conveying a gas such as air, or a liquid, such as water, and a bore 174 for conveying a vacuum or suction, for aspirating liquids. A handheld tool 220 provides controls 222, 224 for controlling the positive pressure of gas or liquid introduced through bore 172, and the negative pressure of suction through bore 174. In accordance with the invention, tool tip 170 is sufficiently malleable to be bent into a convenient orientation to facilitate access to difficult locations, such as the maxillary arch. Tool tip 170 can be used with a pen-style tool body as shown in FIG. 16, or with other tool shapes. The amount of force required to bend tool tip 170 can be predetermined, in order that tool tip 170 is sufficiently stiff to support device 100/100A during normal use, but can be bent by hand to a desired angle.

A material for tool tip 170 is advantageously selected to retain the desired bent shape after a bending force is discontinued. For example, a plastic material such as described elsewhere herein can be used, which will either retain a bent shape at room temperature, or which can be heated, bent, and then cooled to retain a desired shape. Alternatively, a stiffening material can be inserted or molded within tool tip 170 to help maintain a desired bent shape.

Tool tip 170 can be attached to device 100 of the disclosure as described elsewhere here, and as shown in FIG. 17, with bore 172 positioned to eject the gas or liquid to advantageously contact mirror surface 104, further as described elsewhere herein. Aspiration through bore 174 is also positioned in this manner proximate mirror 102, to thereby remove liquid from an environment of the work area.

In FIG. 18, tool 220 includes an internal container 226, for example a cylinder, which is fillable through a port 228 with a liquid or gas, and which can also be pressurized through port 228. A valve 230, which can optionally be a foot operated valve (not shown), releases the pressurized contents of container 226 into hose 232 and to channel 172. In alternative embodiments, container 226 is not present within tool 220, and a hose leads from valve 230 to an external source of pressurized fluid or gas, or a hose leads from channel 172 to an external valve which is connected to an external source of pressurized fluid or gas.

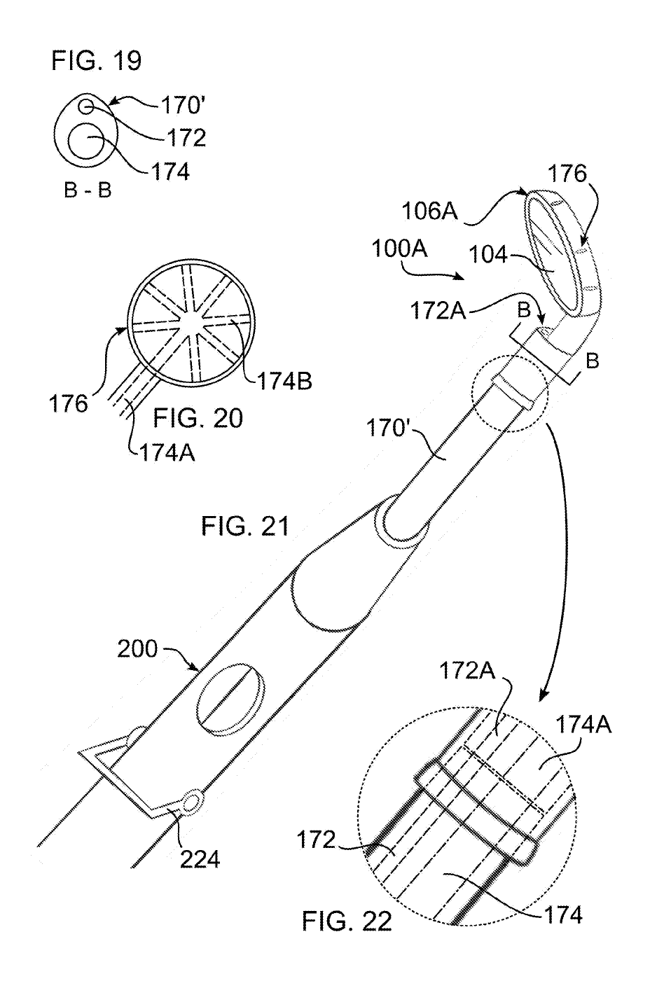

An alternative tool tip attachment device 100A is illustrated in FIGS. 19-21, which includes channels 174A, which transmit suction from channel 174 to the periphery of a mirror supporting frame 106A, when device 100A is mounted upon tool tip 170. In FIG. 21, tool tip 170 is not bent, however it can be bent as described above. FIG. 19 depicts an optional non-circular cross-section of tool tip 170′ which can be mated with a similar cross-section of a socket 180 of device 100A. In this manner, a communicating alignment can be reliably formed between channels 172, 174, and mating channels 172A, 174A of device 100A, as further shown in the enlarged detail of FIG. 22.

Aspiration channels 174B are formed within mirror frame 106A. In FIG. 20, they are depicted as radially extending from a central hub, although any channel configuration which communications vacuum from channel 174A to peripheral aspiration ports 176 can be used. In use, gas or liquid ejected from channel 172A is directed to a surface of mirror 104, whereupon debris is removed, after which the debris and ejected gas or liquid are aspirated into aspiration ports 176 to be suctioned from the work area through tool tip 170, tool 200, and a suction generating device either within tool 220 or connected to tool 220.

Devices 100 (includes 100A) and 200 provide a mirror clip and dental instrument which enables self-cleaning of an indirect vision instrument, and reduces the time required for cleaning, repositioning, or switching hand operated instruments. When device 100 or 200 is firmly attached to a syringe tip 310, air or water is ejected from the syringe and is oriented directly toward the mirror surface 104 to quickly clean the surface. The clip can be rotated 360 degrees to accommodate the clinician's angle preference, yet grips the tool tip with sufficient strength to be used to retract soft tissues such as the patient's lips and cheeks without being displaced from a desired orientation. When the clinician wishes to use the air-water syringe without the mirror, the clip can be removed quickly with the other working hand without a need to reach for an instrument tray. Moreover, once the mirror surface has been cleaned, the fluid can be deflected from the mirror to clean a work area.

Device 100 or 200 of the disclosure can be used without a requirement for a separate fluid connection, and therefore reduces the number of hoses which must be handled and passed into a patient's mouth. The devices are simple and inexpensive in construction, and can therefore be provided as disposable items, reducing sterilization requirements. For the same reasons, they can be provided in a variety of sizes in a kit, so that devices are likely to be available for unusual tool tip sizes.

Devices 100 or 200 can be fabricated using metal, plastic, a composite material, or any other material of sufficient biocompatibility, durability, and strength. Examples include aluminum, titanium, stainless steel, nitinol, nylon, polylactic acid, PVC, polyethylene, PEEK, polycarbonate, Polyetherimide, Polysulfone, Polypropylene, and Polyurethane, or a natural material, such as Gutta-percha. Devices 100, 200 may be fabricated, for example, by casting, molding, carving, 3D printing, or any other method known or hereinafter developed. Separate components of devices 100 or 200 can be connected by any method, including for example adhesive, melting, fasteners, or high frequency welding.

It should be understood that devices 100 and 200 have uses outside of dentistry, to keep mirrors clean in other occupations and industries where a mirror is used for indirect viewing, and where debris is produced during its use. Devices 100 and 200 can further be used together with any known or hereinafter developed mirror lighting system.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

What is claimed is:

1. A device for attachment to an air-water syringe tip, comprising:
a frame;
a mirror surface disposed upon the frame;
a tool grip including a resilient body and a channel extending through the body sized smaller than a diameter of a syringe tip, the channel defining a longitudinal axis; and
a support arm connecting the frame and the tool grip, the frame and the tool grip each connected to the support arm at a predetermined angle to thereby form a predetermined angle between the longitudinal axis of the channel and the mirror surface.

2. The device of claim 1, wherein the tool grip is fabricated from a polymeric material.

3. The device of claim 1, wherein the tool grip further includes a gap extending along the longitudinal axis from the channel to an exterior of the tool grip.

4. The device of claim 1, wherein the predetermined angle between the longitudinal axis of the channel and the mirror surface is between 25 and 70 degrees.

5. The device of claim 1, wherein the predetermined angle between the longitudinal axis of the channel and the mirror surface is about 45 degrees.

6. The device of claim 1, wherein the tool grip is rotatable 360 degrees about the tool tip when the tool tip is inserted into the channel.

7. The device of claim 1, wherein the frame is produced by 3D printing.

8. A system, comprising:
an air-water dental tool tip for replaceable attachment to an air-water dental syringe tool;
a mountable mirror connectable to the air-water dental tool tip, including:
a frame;
a mirror surface disposed upon the frame;
a tool grip including a resilient body and a channel extending through the body sized smaller than a diameter of a syringe tip, the channel defining a longitudinal axis; and
a support arm connecting the frame and the tool grip, the frame and the tool grip each connected to the support arm at a predetermined angle to thereby form a predetermined angle between the longitudinal axis of the channel and the mirror surface.

9. The device of claim 8, wherein the system includes a plurality of air-water dental tool tips of differing tool tip diameters, and a plurality of mountable mirrors, at least one mountable sized for mounting to each of the plurality of tool tips by having a channel sized smaller than the diameter of the tool tip.

10. The system of claim 8, wherein the tool grip is fabricated from a polymeric material.

11. The system of claim 8, wherein the tool grip further includes a gap extending along the longitudinal axis from the channel to an exterior of the tool grip.

12. The system of claim 8, wherein the predetermined angle between the longitudinal axis of the channel and the mirror surface is between 25 and 70 degrees.

13. The system of claim 8, wherein the predetermined angle between the longitudinal axis of the channel and the mirror surface is about 45 degrees.

14. The device of claim 8, wherein the tool grip is rotatable 360 degrees about the tool tip when the tool tip is inserted into the channel.

15. The system of claim 8, wherein the frame is produced by 3D printing.

16. The system of claim 8, wherein the frame is formed from a material selected from the group consisting of polylactic acid, PVC, polyethylene, PEEK, polycarbonate, Polyetherimide, Polysulfone, Polypropylene, and Polyurethane.

17. A method of cleaning a dental mirror, comprising:
providing a device attachable to an air-water syringe tip, the device including a frame;
a mirror surface disposed upon the frame;
a tool grip including a resilient body and a channel extending through the body sized smaller than a diameter of a syringe tip, the channel defining a longitudinal axis;
a support arm connecting the frame and the tool grip, the frame and the tool grip each connected to the support arm at a predetermined angle to thereby form a predetermined angle between the longitudinal axis of the channel and the mirror surface.

18. The method of claim 17, wherein the tool grip is fabricated from a polymeric material.

19. The method of claim 17, wherein the tool grip further includes a gap extending along the longitudinal axis from the channel to an exterior of the tool grip.

20. The method of claim 1, wherein the gap is resiliently expanded when the tool tip is inserted into the tool grip.
